(19) United States
(12) Patent Application Publication
(10) Pub. No.: US 2008/0119499 A1
(43) Pub. Date: May 22, 2008

(54) Compositions Havinge Improved 2-Photon Absorbance For Nonlinear Applications

(75) Inventors: Andrew N. Shipway, Jerusalem (IL); Nimer Jaber, Abu Ghosh (IL); Moshe Greenwald, Tel-Aviv (IL); Michael Grishko, Haifa (IL); Ariel Litwak, Ramat Hasharon (IL)

Correspondence Address: BROWDY AND NEIMARK, P.L.L.C.;624 NINTH STREET, NW, SUITE 300, WASHINGTON, DC, 20001-5303, US

(73) Assignee: MEMPILE INC., WILMINGTON (DE)

(21) Appl. No.: 11/813,820

(22) PCT Filed: Jan. 12, 2006
(86) PCT No.: PCT/IL06/00052

§ 371 Date: Jul. 12, 2007

Related U.S. Application Data
(60) Provisional application No. 60/643,106, filed on Jan. 12, 2005.

(51) Int. Cl. H01L 51/00; A61K 31/275

(52) U.S. Cl. 514/277; 514/520; 558/402; 257/40; 257/E51.02

(57) ABSTRACT

There are provided conjugated systems of formula (I) based on stilbene derivatives serving as improved Donor-Acceptor-Donor compounds and their use in non linear optics including chromophoric optical medium of a 3-dimensional optical memory.

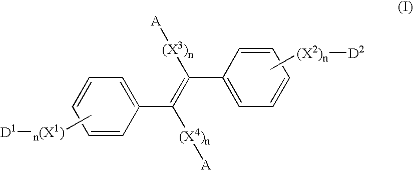

### FIELD OF THE INVENTION

This invention relates to improved chromophores having strong 2-photon absorbance that undergo photoinduced isomerization processes.

### BACKGROUND OF THE INVENTION

Recent developments in the field of electro-optic applications are based on development of chromophores having strong 2-photon absorbance. Applications which benefit from utilization of chromophores having strong 2-photon absorbance include optical limiting, data storage, 3D microfabrication and telecommunications. Chromophores having a general structural formula consisting of a “Donor-Acceptor-Donor” (DAD) structure tend to have a strong 2-photon absorbance. Such compounds were previously used as the active medium of a 3-dimensional optical memory (WO 03/070,689).

Stilbenes, and bis-donor-disubstituted stilbenes in particular, have an excited state that is characterized by strong charge-transfer from the donors to the center of the molecule, the double bond. Resonance forms that can be drawn by “pushing electrons” in a diaminostilbene compound give a structures that correspond to excited-states that can be calculated (Wang et al. J. Mater. Chem. (2001) 11, 1600).

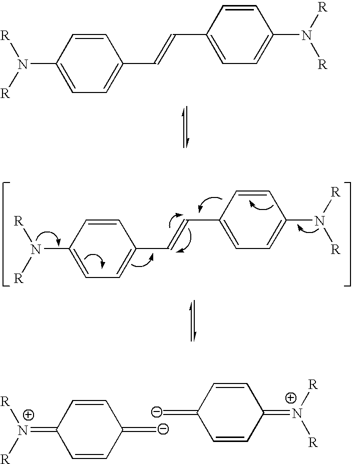

This charge transfer gives the molecule strong third-order nonlinear optical activity, since it causes a significant second harmonic component in the perturbance of the chromophore's electrons upon interaction with a fluctuating electric field. In addition, it should be noted that in the charge-transferred form, the central double-bond is weakened or does not exist. This observation explains the efficient photoisomerization of the molecule, and why isomerization is faster with stronger charge-transfer donors.

It should be noted that substitution of the hydrogens on the double bond in stilbenes with chemical groups being stronger electron acceptors should be carefully done since the entire electronic layout of the substituted stilbene which is a fully conjugated system may lead to the destruction of the stilbene upon absorbance of energy. A balanced approach should be taken in case it is desirous to increase the donor-acceptor features of the system by using less strong donors and stronger acceptors.

### SUMMARY OF THE INVENTION

According to the present invention novel compounds having strong 2-photon absorbance are provided as well as their use in various electro-optic applications.

Thus the present invention provides compounds of formula (1)

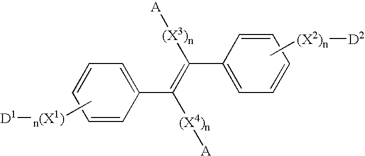

wherein n is independently 0, 1 or 2; X¹, X², X³ and X⁴ are conjugated groups, each of which is independently selected from (i) 5 or 6-membered aromatic moieties which may also contain one, two or three heteroatoms selected from N, O or S, and (ii) C₂-C₅ alkenylenes or C₂-C₅ alkynylenes or their heteroatom chain analogs which optionally may comprise a N, O or S atom in the chain; one or both of said X¹ and X² may be bound to the 3, 4 or 5 positions of the phenyl ring, or one or both of said X¹ and X² may form a fused ring with the phenyl ring in the 3-4 or 4-5 positions; D¹ and D² are electron donor moieties independently selected from —C₁₋₄alkyls, —OC₁₋₄alkyl, —SC₁₋₄alkyl and, —C₁₋₄OH, thiols and their salts, NR′R″, R′ and R″ being independently hydrogen or C₁₋₄alkyl, biphenyls, and heteroaromatics selected from five- and six-membered rings having one or two heteroatoms selected from N, O or S; A is an electron acceptor moiety selected independently from pyridinium, ammonium salts, C₂₋₅alkenyl or C₂₋₅alkynyl groups, azobenzenes, C₁₋₄CN, halides, C₁₋₆COOH or their C₁₋₆esters, or C₁₋₄NO₂ compounds.

Preferred are compounds wherein D¹ and D² groups in the Donor system may each independently be comprised of an NR′R″, R′ and R″ being independently hydrogen or C₁₋₄alkyl, —C₁₋₄OH, —OC₁₋₄alkyl groups; X¹ and X² groups are preferably each independently optionally substituted phenyl, pyridine, pyrrole, imidazole, triazole, thiophene, furan with the substituents being —C₁₋₄alkyls, —OC₁₋₄alkyl.

The A groups of the Acceptor system are preferably independently cyano or nitro; X³ and X⁴ are preferably each independently C₂₋₅alkenyl or C₂₋₅alkynyl, or their heteroatom chain analogs which optionally may comprise a N, O or S atom in the chain.

It should be understood that it is possible for the Acceptor system (A—(X³)ₙ— or A—(X⁴)ₙ) or Donor system (D¹—(X¹)ₙ— or (D¹—(X²)ₙ) to have oily one conjugation, e,g, one n=0 in each system.

The present invention thus provides compounds of formula (2)

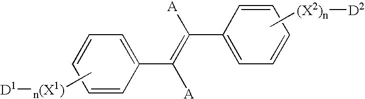

wherein n is independently 0, 1 or 2; X¹, X² are conjugated groups independently selected from (i) 5 or 6-membered aromatic moieties which may also contain one, two or three heteroatoms selected from N, O or S, and (ii) C₂-C₅ alkenylenes or C₂-C₅ alkynylenes or their heteroatom chain analogs which optionally may comprise a N, O or S atom in the chain; one or both of said X¹ and X² may be bound to the 3, 4 or 5 positions of the phenyl ring, or one or both of said X¹ and X² may form a fused ring in the 3-4 or 4-5 positions; D¹ and D² are electron donor moieties independently selected from —C₁₋₄alkyls, —OC₁₋₄alkyl, —SC₁₋₄alkyl and, —C₁₋₄OH, thiols and their salts, NR′R″, R′ and R″ being independently hydrogen or C₁₋₄alkyl, biphenyls, and heteroaromatics; A is an electron acceptor moiety independently selected from pyridinium, ammonium salts, C₂₋₅alkenyl or C₂₋₅alkynyl groups, azobenzenes, C₁₋₄CN, halides, C₁₋₆COOH or their C₁₋₆esters, or C₁₋₄NO₂, compounds.

Preferred are compounds of formula (1), wherein X¹ and X² are independently conjugated groups selected from 5 or 6-membered aromatic moieties which may also contain one, two or three heteroatoms selected from N, O or S; said moieties may be fused or bonded to the phenyl ring; D¹ and D² are independently alcohol, alkoxy or amino and A is cyano or nitro.

The present invention thus further provides compounds of formula (3):

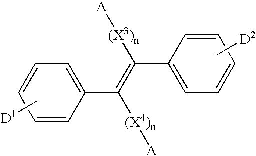

wherein n is independently 0, 1 or 2; X³, X⁴ are conjugated groups independently selected from (i) 5 or 6-membered aromatic moieties which may also contain one, two or three heteroatoms selected from N, O or S, and (ii) C₂-C₅ alkenylenes or C₂-C₅alkynylenes or their heteroatom chain analogs which optionally may comprise a N, O or S atom in the chain; D¹ and D² are electron donor moieties independently selected from —C₁₋₄alkyls, —OC₁₋₄alkyl, —SC₁₋₄alkyl and, —C₁₋₄OH, thiols and their salts, NR′R″, R′ and R″ being independently hydrogen or C₁₋₄alkyl, biphenyls, and heteroaromatics; A is independently an electron acceptor moiety selected from pyridinium, ammonium salts, C₂₋₅alkenyl or C₂₋₅alkynyl groups, azobenzenes, C₁₋₄CN, halides, C₁₋₆COOH or their C₁₋₆esters, or C₁₋₄NO₂ compounds.

Preferred are compounds of formula (3), wherein D¹ and D² are independently alcohol or amino and A is cyano or nitro; X³, X⁴ are C₂-C₅alkenylenes or C₂-C₅alkynylenes optionally having one, two or three heteroatoms selected from N, O or S atoms and n=1.

Specific preferred compounds according to the invention are the following:
2-(4′-Methoxy-biphenyl-4-yl)-3-(4-methoxymethyl-phenyl)-but-2-enedinitrile
2-(4′-Formyl-biphenyl-4-yl)-3-(4-methoxymethyl-phenyl)-but-2-enedinitrile
2-(4′-Dimethylamino-biphenyl-4-yl)-3-(4-methoxymethyl-phenyl)-but-2-enedinitrile
2,3-Bis-(4′-methoxy-biphenyl-4-yl)-but-2-enedinitrile
2,3-Bis-(4′-methyl-biphenyl-4-yl)-but-2-enedinitrile

The present invention is further directed to compounds of formulae (1)-(3) above used in nonlinear optics, for example as specialty dyes, reference standards, optical limiters, as components in microfabriation systems, for nanotechnological devices, and for targeted medical therapeutic applications. In particular, these compounds may be used as the active medium of a 3-dimensional optical memory.

The present invention is further directed to devices or formulations comprising any one of the compounds (1)-(3). Examples are specialty dyes, reference standards, optical limiters, microfabriation systems, nanotechnological devices, devices or pharmaceutical compositions for use in targeted medical therapeutic applications. In particular, provided are 3-dimensional optical memory devices of the kind disclosed in WO 01/73,769 and WO 03/070,689, which are incorporated herein by reference, comprising the compounds of the invention.

The present invention is also directed to a process for the preparation of the compounds of any one of formulae (1)-(3).

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 demonstrates conjugation pathways in substituted stilbenes which may lead to either continued charge transfer to all parts of the stilbene or to the loss of an ion resulting in an oxidative decomposition of the stilbene.

FIG. 2 illustrates process for the preparation compounds of formula (1) according to the present invention

FIG. 3 illustrates several other synthetic routes to compounds of the invention.

FIG. 4 illustrates comparative ultra violet spectrum of several compounds of formula (1) demonstrating the higher yield of interconversion from one isomeric form to the other as a result of the increase of the conjugation in the compound of formula (1).

### DETAILED DESCRIPTION OF EXEMPLARY EMBODIMENTS

As mentioned the present invention is directed to compounds of formulae (1)-(3) having strong 2-photon absorbance due to charge transfer through an extended conjugated systems in these compounds. The compounds of formulae (1)-(3) have an excited state that is characterized by strong charge transfer from the donors to the acceptors at the center of the compound. As a result of the strong charge transfer, such compounds undergo upon irradiation, isomerization at a higher rate compared to compounds which do not have an extended conjugated system. The extended conjugated systems are 5 or 6 aromatic, optionally heteroaromatic groups which may be either fused or directly bonded to an existing rich π-electrons system, or may be an alkynylene or alkenylene groups conjugated to the rich π-electrons system.

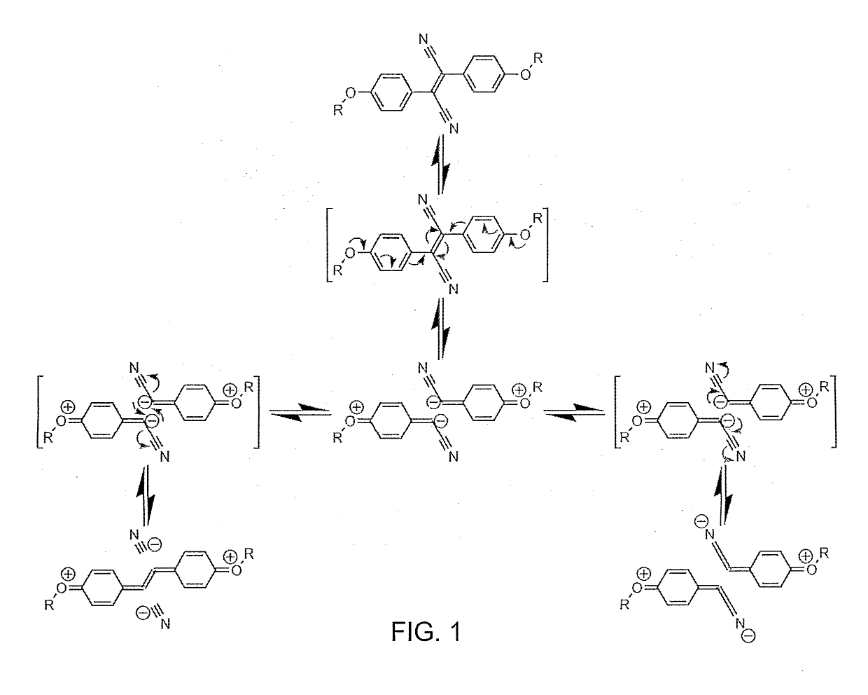

In general, aryl substituted ethenes having substituted aromatic groups serving as electron donors (D) form a DAD system since the “ethenic” bond, the π-electrons, may serve as an acceptor (A) moiety. In such a system, plain electronic considerations dictate the efficiency of the system. An amine substituted aromatic ring is a better donor group than an alkoxy substituted aromatic group. However, in case the hydrogens on the “ethenic” double bond are substituted by acceptor moieties, a modified DAD system evolves where it is possible to use weaker donor moieties such as an alkoxy where the acceptor is a nitrile or nitro group. Hence a α,α′-dimethoxy-stilbene-dicyano (DMSDC) has charge transfer properties similar to those of α,α′-diaminostilbene, since the weaker donor properties of the alkoxy groups on the aromatic groups is compensated by the existence of the cyano substituents on the double bond. However, the acceptors in the DMSDC can take part in the conjugation, an issue which makes the system more complicated. This can be observed in FIG. 1 for DMSDC. In this figure, the DMSDC 10 may exist in a transition state 20 which may lead to a di-zwiterionic structure 30. This latter structure 30 may further undergo to two transition states 40 and 50 which may lead to the formation of two ionic resonance structures 60 and 70, respectively. In structure 60, there is a loss of cyanide ion whereas in structure 70 there is a possibility of rearrangement in the stilbene to a form n isomeric form.

One manner of optimizing the chromophores and enhancing their 2-photon cross-section raising the rate of isomerization is to increase the conjugation length. Such an increase in the conjugation, which may be termed as chromophores having extended conjugated systems, may be done for either both the Donor system and the Acceptor system in the same chromophore or an improved chromophore may have either a more conjugate Donor or a more conjugated Acceptor. This may be seen in the following exemplary Scheme A, which shows a more conjugated Donor system being a biphenyl substituted by an alkoxy group rather than a phenyl substituted by an alkoxy group.

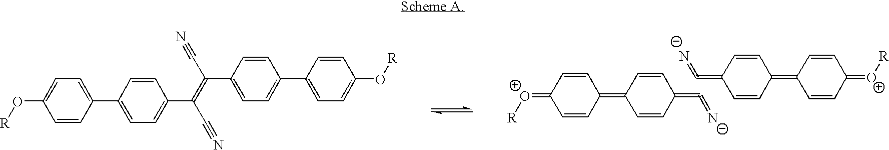

Scheme B shows another examplary more conjugated Acceptor system being an acetylene-nitrile rather than a nitrile as shown.

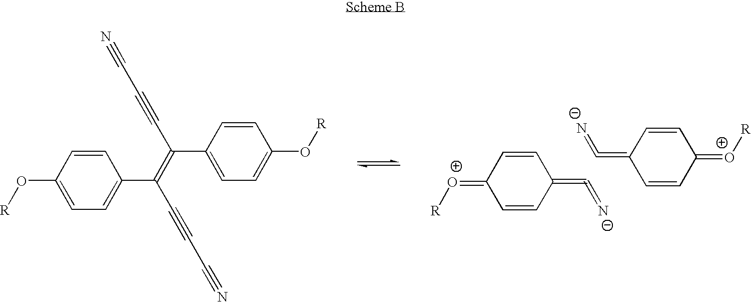

An improved chromophore having an improved more conjugated Acceptor and Donor systems may schematically be represented as in the following Scheme C:

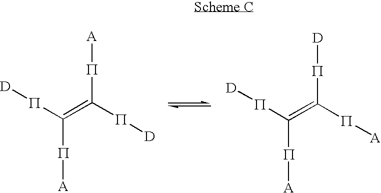

Such a system is a quadrupolar DADA system, where the π-spacers may be aromatic, alkenyl, alkynyl, or a combination thereof. Scheme C clearly shows the large change in quadrupole that occurs upon isomerization, i.e. the transfer from “E” to “Z”. This is important for the photochromic applications. The change in such a system may be greater than the change induced in other photochromes such as stilbenes or even substituted stilbenes which are D—A—D systems upon photoisomerization.

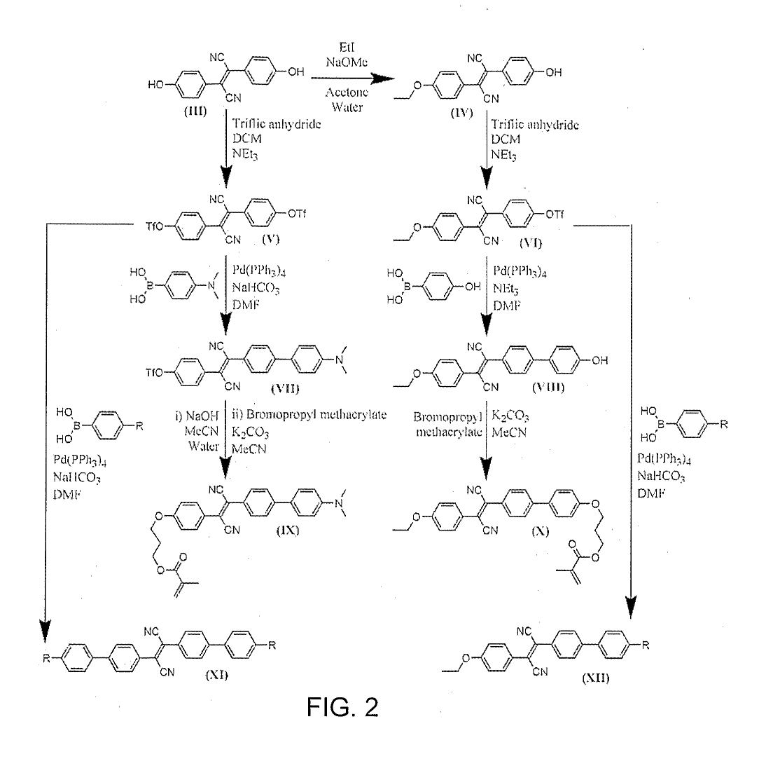

The improved chromophores of the present invention offer significantly improved 2-photon absorption over their analogs where X¹, X², X³ and X⁴ are not present, and they are capable of displaying trans-cis photochromism. It should be noted that in a maimer similar to that disclosed in WO 03/070,689, the compounds of formulae (1), (2) or (3) of the present invention may be covalently attached to polymerizable groups, leading to monomers that allow the production of plastics with photochromic and nonlinear optical properties. Such compounds are specifically shown in FIG. 2 as compounds (LX) and (X). In these compounds, there is only one extended more conjugated Donor, where a methyl(meth)acrylate moiety is attached either to the extended more conjugated Donor or the regular Donor.

The compounds of the present invention may be utilized in applications requiring improved 2-photon absorption. One example of an application for these materials is data storage, where the compounds of the present invention may serve as more efficient chromophores for data storage such as 3-dimensional optical data storage disclosed in WO 01/73,769 and WO 03/070,689. It is also noted that the fluorescence of these molecules tends to be highly dependant on their microenvironment, and so they may also be utilized as viscosity sensors.

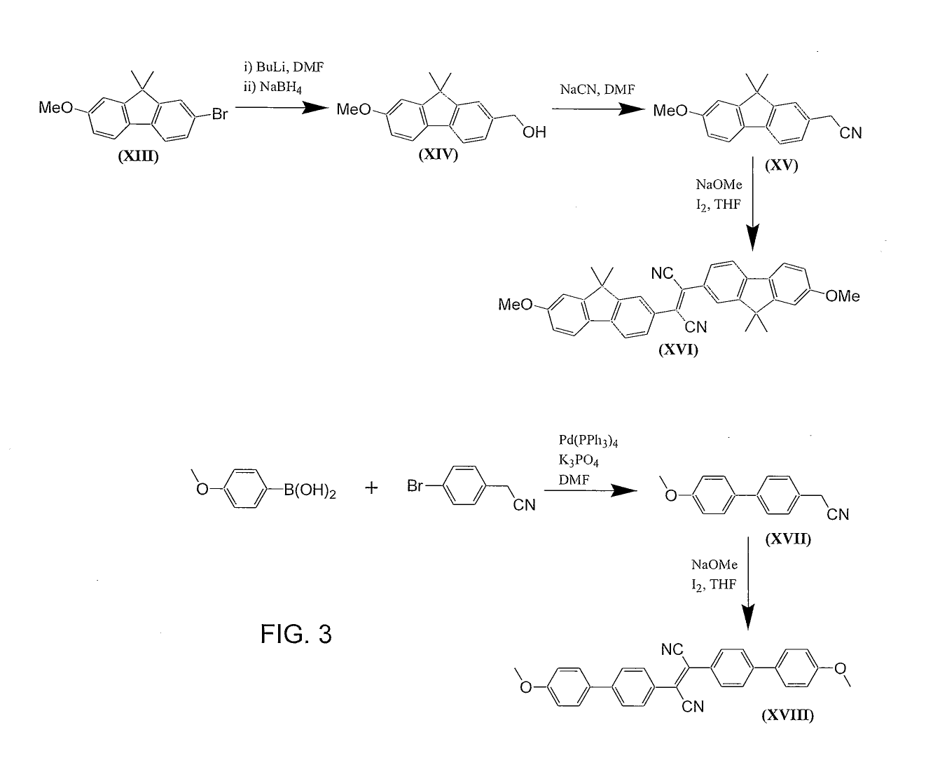

Some compounds of the present invention may be prepared as schematically shown in FIG. 2 (detailed routes given below). The synthesis of compounds (III) and (IV) can be found in WO 03/070,689. Briefly, monoalkylation of α,α′-bis(4-hydroxy)-trans-dicyanostilbene (III) leads to the formation of (IV). The two compounds (III) and (IV) are triflated to lead to the key intermediates (V) and (VI) (see examples). The biaryl moieties are then formed by Pd-catalyzed cross-coupling (Suzuki) methodology. The general structures (XI) and (XII) have been demonstrated with R═OMe and with R═NMe₂. Another route that has been verified is the reaction of compound (V) with 4-methoxtphenyl boronic acid, under Suzuki conditions, to afford an analog to (VII). The structures of embodiments presented have been verified by NMR and mass spectrometry. Other synthetic routes to compounds of the invention are shown in FIG. 3. These routes can be considered to be general for similarly functionalized chromophores.

### EXAMPLES

The synthesis schemes and the compounds referred to in the Examples below can be found in FIGS. 2 and 3.

### Example 1

### Synthesis of Compound (V)

To an oven-dried, argon-flushed 500 mL reaction vessel, 10 g of compound (III) (see FIG. 2) and 21.2 g of triethylamine were added. The mixture was stirred at ambient temperature for 5 minutes as the color became dark red, then it was cooled to 0° C. Triflic anhydride (56.4 g) was added dropwise over a period of 30 minutes, then the reaction temperature was allowed to rise to ambient and the brown-black mixture was stirred for 24 hours. The mixture was filtered and the solid was washed with water then dried to give (V) in 78% yield.

### Example 2

### Synthesis of Compound (VI)

To a three-neck round bottom flask connected to a condenser compound (IV) was added (10 grams, 0.034 moles), dichloromethane (200 mL), triethylamine (20 mL) and triflic anhydride (20 mL). The mixture was magnetically stirred for 24 hours at room temperature and monitored by TLC (DCM:PE (2:4)). Dichloromethane (300 mL) was added and the mixture was washed twice with a diluted potassium hydroxide solution (300 mL), then with a diluted hydrochloric acid solution (300 mL) and finally with water (300 mL). The organic phase was dried over magnesium sulfate, filtered and the solvent was evaporated to give 14 grams of crude material. The crude material was purified by flash chromatography under silica and DCM:PE (1:1) as the eluent, to give 1.7 grams (0.004 moles, 11.8% yield). The resulting compound (VI) had the following ¹H-NMR (CDCl₃) spectrum:

¹H-NMR (CDCl₃) δ (ppm)=7.46 (m, 2H), 7.28 (m, 2H), 7.21 (m, 2H), 6.81 (m, 2H), 4.05 (q, 2H), 1.42 (t, 3H).

### Example 3

### Synthesis of Compound (VII)

Compound (V) (255 mg), DMF (3 ml, dry), Pd(PH₃)₄ (20 mg) and 4-dimethylamino phenylboronic acid (111 mg) were added to a dry, argon-purged reaction vessel. NaHCO₃ (281 mg) was added and the temperature was raised to 80° C. The reaction was continued with stinting under argon for 20 hours, after which it was cooled and then quenched with HCl (3 mL) and water (10 mL). The mixture was filtered to give a solid that was subjected to column chromatography on silica gel (1:1 chloroform:hexane thenpure chloroform) to yield the desired product (55 mg, 20% yield). The resulting compound (VII) had the following ¹H-NMR (CDCl₃) spectrum:

¹H-NMR (CDCl₃) δ (ppm)=3.09 (6H, s), 6.9 (2H, br) 7.53 (2H, m), 7.64 (2H, m), 7.77 (2H, m), 7.97 (4H, m).

### Example 4

### Synthesis of Compound (VIII)

Compound (VI) (1.73 g), DMF (20 ml, dry), Pd(PH₃)₄ (95 mg) and 4-hydroxyphenylboronic acid (565 mg) were added to a dry, argon-purged reaction vessel. triethylamine (1.7 mL) was added and the temperature was raised to 80° C. The reaction was continued with stirring for 20 hours, after which the DMF was removed under vacuum. The crude product was subjected to column chromatography on silica gel (chloroform, then 2% MeOH in chloroform) to yield the desired product (850 mg).

### Example 5

### Synthesis of Compound (IX)

Compound (VII) (25 mg) was dissolved in a mixture of DMF (3 ml), MeOH (1.5 ml) and 3M NaOH (1.5 mL) to give a red solution. The mixture was heated to reflux for 4 hours, and then was cooled and 1M HCl (4.5 mL) was added. Pyridine (1 mL) was added, and then the solvents were removed under vacuum. The residue was washed with water, then was subjected to column chromatography on silica gel (eluting with chloroform, then 2% MeOH in chloroform) to give the deprotected product, whose identity was confirmed by mass spectroscopy.

˜2 mg of the deprotected product was dissolved in ˜3 ml of MeCN and potassium carbonate (˜100 mg) and 3-bromopropyl methacrylate (˜100 mg) were added. The mixture was stirred at ambient temperature for 20 hours, and then was filtered. The solvent was removed by evaporation, and then the residue was purified by preparative HPLC to give a sub-milligram quantity of the orange-red product. The resulting compound (IX) had the following mass spectrum:

M.S (EI+): 530 (MK⁺, 100%), 515 (M Na⁺, 19%), 491 (M⁺, 21%)

### Example 6

### Synthesis of Compound (X)

Compound (VIII) (850 mg) was dissolved in a mixture of DMF (10 mL) and MeCN (20 mL), then potassium carbonate (1 g) and 3-bromopropyl methacrylate (1 g) were added. The reaction was heated to 60° C. for 5 hours, then was cooled and filtered, and the solids were washed with chloroform. The combined filtrate was condensed under vacuum, then MeOH (30 mL) was added and the mixture was put in the freezer for 2 hours. The product was collected by filtration (555 mg). The resulting compound (X) had the following ¹H-NMR (CDCl₃) spectrum:

¹H-NMR (CDCl₃) δ (ppm)=7.90 (m, 4H), 7.13 (m, 2H), 7.62 (m, 2H), 7.05 (m, 4H), 6.17 (m, H), 5.62 (m, H), 4.42 (t, 2H), 4.16 (m, 4H), 2.25 (tt, 2H), 2.00 (s, 3H), 1.50 (t, 3H).

### Example 7

### Synthesis of (XI)

A. Preparation of the precursor (4′-Methoxy-biphenyl-4-yl)-acetonitrile: 4-bromophenylacetonitrile (2.35 g, 12.0 mmol), 4-methoxyphenylboronic acid (2.00 g, 13.2 mmol, 1.1 eq), K₂PO₄ (5.08 g, 23.9 mmol, 2 eq) were heated up in DMF (33 cc) to 95° C., under Ar atmosphere stream. After 20 minutes, during which water residues were removed, Pd(PPh₃)₄ (0.27 g, 2% eq) was added and reaction mixture was stirred for 3.5 hrs. Cool to room temperature and filter. To filtrate add chloroform (100 cc) wash with water (5×100 cc), dry (MgSO₄), filter and evaporate to yield 2.88 g solid product (85% pure by HPLC). After recrystallization from hexane:chloroform:2-propanol (205:11:9 cc) 0.74 g of light yellowish bright powder was obtained, y=28% (99% pure by HPLC). M.S (EI+): 223 (M⁺, 100%), 208 ([M−Me]⁺, 26%), 180 (MeO—Ph—Ph—H⁺, 22%).
B. Coupling of the precursor to obtain 2,3-Bis-(4′-methoxy-biphenyl-4-yl)-but-2-enedinitrile: A partially dissolved mixture of (4′-Methoxy-biphenyl-4-yl)-acetonitrile (0.74 g, 3.3 mmol) and iodine (0.925 g, 1.1 eq) in MTBE (8.4 cc) was stirred at −20° C., under Ar atmosphere, for 3 minutes. Sodium methoxide (25% solution in methanol, 1.7 cc, 2.2 eq) was added dropwise over 30 minutes. The orange-brown reaction mixture was allowed to reach room temperature, over 30 minutes, and stirred for additional 30 minutes. Reaction mixture was filtered and the solid was washed thoroughly with MTBE. The solid was transferred to 0.1 M sodium thiosulfate solution stirred, filtered again and washed with much water. A total of 0.53 g of 2,3-Bis-(4′-methoxy-biphenyl-4-yl)-but-2-enedinitrile were obtained at ˜90% purity (main impurity is starting material), y=33%. Adequate purity may be obtained after treating with hot P.E. and hot MeOH.

### M.S (EI+): 442 (M⁺, 100%), 223 (MeO—Ph—Ph—CH₂CN⁺, 12%).

### Example 8

### Synthesis of Compound (XII) (R═OMe)

To a well dry three-neck 100 ml round bottom flask connected to a condenser and under argon atmosphere, 10 mL of fresh distilled DMF was added. Then Compound (VI) (0.57 grams, 1.3 mmoles), 4-anisylboronic acid (0.21 grams, 1.38 mmoles), potassium phosphate tribasic (0.42 grams, 2 mmoles) and tetrakis-(triphenylphosphin)-palladium (0) (0.023 grams, 0.02 mmoles) were added. The mixture was flushed with argon while stirring. The reaction mixture was heated to 110° C. for 20 hours. TLC test showed remainder starting material, and additional 4-anisylboronic acid (0.1 grams, 0.66 mmoles) was added, and the mixture was heated at 110° C. for additional 4 hours. The solvent was removed under vacuum, and chloroform (10 ml) was added. The organic solution was washed twice with diluted HCl (10 ml) and then dried over magnesium sulfate. The crude material (0.7 grams) was purified by flash chromatography under silica and DCM:PE (1:1) as the eluent, to give 120 mg (0.31 mmoles, 24.3% yield). The resulting compound (XII) had the following ¹H-NMR (CDCl₃) spectrum:

¹H-NMR (CDCl₃) δ (ppm)=7.88 (m, 2H), 7.84 (m, 2H), 7.70 (m, 2H), 7.59 (m, 2H), 7.02 (m, 4H), 4.13 (q, 2H), 3.87 (s, 3H), 1.47 (t, 3H).

### Example 9

### Synthesis of (XII) (R═NMe₂)

To a well dry three-neck 50 ml round bottom flask connected to a condenser and under argon atmosphere, 10 ml of fresh distilled DMF was added. Then compound (VI) (0.5 grams, 1.18 mmoles), 4-dimethylaminophenylboronic acid (0.234 grams, 1.42 mmoles), potassium phosphate tribasic (0.5 grams, 2.35 mmoles) and tetrakis-(triphenylphosphin)-palladium (0) (0.027 grams, 0.023 mmoles) were added. The mixture was flushed with argon while stirring. The reaction mixture was heated to 115° C. for 24 hours. The solvent was removed under vacuum, and chloroform (10 ml) was added. The organic solution was washed twice with water (10 ml) and then dried over magnesium sulfate. The crude material (0.6 grams) was purified by flash chromatography under silica and chloroform:acetone:ammonium hydroxide (25:1:1) as the eluent, to give 40 mg (0.1 mmoles, 8.6% yield). The resulting compound (XII) had the following ¹H-NMR (CDCl₃) spectrum:

¹H-NMR (CDCl₃) δ (ppm)=7.91 (m, 2H), 7.87 (m, 2H), 7.74 (m, 2H), 7.63 (m, 2H), 7.05 (m, 2H), 6.9 (br m, 2H), 4.16 (q, 2H), 3.09 (s, 6H), 1.50 (t, 3H).

### Example 10

### Synthesis of (XII) (R═CHO)

To a well dry three-neck 100 ml round bottom flask connected to a condenser and under argon atmosphere, 10 ml of fresh distilled DMF was added. Then compound (VI) (0.5 grams, 1.18 mmoles), 4-formylphenylboronic acid (0.27 grams, 1.8 mmoles), potassium phosphate tribasic (0.48 grams, 2.26 mmoles) and tetrakis-(triphenylphosphin)-palladium (0) (0.027 grams, 0.0235 mmoles) were added. The mixture was flushed with argon while stirring. The red colored reaction mixture was heated to 100° C. for 20 hours. The solvent was removed under vacuum, and chloroform (10 ml) was added. The organic solution was washed twice with diluted HCl (10 ml) and then dried over magnesium sulfate. The crude material was purified by flash chromatography under silica and DCM:PE (1:1) as the eluent, to give 200 mg (0.53 mmoles, 44.8% yield). The resulting compound (XII) had the following ¹H-NMR (CDCl₃) spectrum:

¹H-NMR (CDCl₃) δ (ppm)=10.09 (s, H), 8.01 (m, 2H), 7.93 (m, 2H), 7.87 (m, 2H), 7.81 (m, 2H), 7.79 (m, 2H), 7.03 (m, 2H), 4.13 (q, 2H), 1.47 (t, 3H).

### Example 11

### Synthesis of (XIII)

To a stirred solution of 2-bromo-7-acetyl-9,9-dimethyl-fluorene (9 g, 28 mmol) in the ethanol-free chloroform (250 mL) was added (under N₂ at 0° C.) portion-wise solid 70% MCPBA (1.3 eq., 33 mmol, 8.0 g). The mixture was allowed to warm slowly to ambient and stirred for 3 days in the dark. The completion of the reaction was checked by HPLC. Aq. 10% NaHCO₃ (150 mL) was added and the mixture was stirred vigorously for 40 min. The phases were separated, the aqueous layer was extracted again with chloroform and the combined organic extracts were washed with brine, dried over MgSO₄ and evaporated. The crude product (9.1 g) was taken forward without further purification. The resulting product had the following ¹H-NMR (CDCl₃) spectrum:

¹H-NMR (CDCl₃) δ (ppm)=7.66 (d, 1H, J=7.5 Hz), 7.55 (d, 1H, J=2.0 Hz), 7.53 (d, 1H, J=7.5 Hz), 7.46 (dd, 1H, J₁=7.5 Hz, J₂=2.0 Hz), 7.15 (d, 1H, J=2.5 Hz), 7.07 (dd, 1H, J₁=7.5 Hz, J₂=2.5 Hz), 2.33 (s, 3H), 1.47 (s, 6H); ¹³C-NMR (CDCl₃) δ (ppm)=170.3, 156.4, 155.4, 151.2, 138.1, 136.5, 130.9, 126.8, 122.0, 121.7, 121.4, 121.3, 116.9, 47.9, 27.6, 21.9

The crude 2-acetoxy-7-bromo-9,9-dimethyl-fluorene (9.1 g, ˜27 mmol) was dissolved in EtOH (120 mL) and treated portion-wise with solid NaOH (˜3 eq., 3.3 g). The hydrolysis reaction was completed within 2 hr at ambient (TLC, Hexane/EtOAc 4:1 and HPLC monitoring). Mel (˜2.5 eq., 9.6 g) was added and the mixture was stirred overnight at ambient. More MeI and/or NaOH were added to complete the alkylation (0.5 eq. of each material). When no remaining phenol could be detected (TLC and HPLC monitoring), the reaction mixture was poured slowly into cold water and neutralized with dilute HCl. The mixture was extracted 3 times with ether, the combined organic extracts were washed with brine, dried over MgSO₄ and evaporated. The pure product was isolated by chromatography (Hexane/EtOAC 10:1). Yield 8.0 g (26.4 mmol, 92% after 2 steps). The resulting compound (XIII) had the following ¹H-NMR (CDCl₃) spectrum:

¹H-NMR (CDCl₃) δ (ppm)=7.59 (d, 1H, J=8.5 Hz), 7.53 (d, 1H, J=1.5 Hz), 7.50 (d, 1H, J=8.0 Hz), 7.46 (dd, 1H, J₁=8.5 Hz, J₂=1.5 Hz), 6.96 (d, 1H, J=2.5 Hz), 6.89 (dd, 1H, J₁=8.0 Hz, J₂=2.5 Hz), 3.88 (s, 3H), 1.47 (s, 6H).

### Example 12

### Synthesis of (XIV)

To the stirred solution of 2-methoxy-7-bromo-9,9-dimethyl-fluorene (compound (XIII)) (8.0 g, 26.4 mmol) in dry THF (150 mL) under N₂ at −78° C. was added drop-wise n-BuLi (1.6M solution in hexane, 1 eq.) during 20 min. The mixture was stirred for 30 min, checking the completion of metallation by HPLC. Dry DMF (1.4 eq., 37 mmol, 2.7 g), mixed with THF (6 mL), was added drop-wise, the mixture was stirred for 1 hr at −78° C., then allowed to warm slowly to ambient and stirred for another hour. The red color of initially formed anion faded upon completion of the reaction (HPLC monitoring). The reaction mixture was cooled to 0° C., quenched with 3M HCl, stirred for 20 min, diluted with water and extracted twice with ether. The combined organic extracts were washed with 10% NaHCO₃, brine and evaporated under reduced pressure. The crude aldehyde was dissolved in EtOH (100 mL) and treated portion-wise (foaming!) under N₂ with solid NaBH₄ (˜2 eq., 50 mmol, 1.9 g). The reaction mixture was stirred for 2 hr at ambient, with TLC (Hexane/EtOAc 4:1) or HPLC monitoring. When the reaction was completed, most of EtOH was evaporated (Rotavapor, bath temp <40° C.), then the mixture was slowly poured into 10% NaHCO₃ (150 mL) and stirred vigorously for 20 min. The mixture was diluted with water, extracted with ether (3×80 ml), the combined organic extracts were washed with brine, dried over MgSO₄ and evaporated. The crude product was purified by flash chromatography (Hexane/EtOAC 20:1, then 10:1, finally 4:1 eluting the desired product). The yield of pure 7-(2-methoxy-9,9-dimethyl)-fluorenyl carbinol was 4.5 g. The resulting compound (XIV) had the following ¹H-NMR (CDCl₃) spectrum:

¹H-NMR (CDCl₃) δ (ppm)=7.61 (d, 1H, J=8.0 Hz), 7.60 (d, 1H, J=8.0 Hz), 7.41 (d, 1H, J=1.0 Hz), 7.29 (dd, 1H, J₁=8.0 Hz, J₂=1.0 Hz), 6.97 (d, 1H, J=2.5 Hz), 6.88 (dd, 1H, J₁=8.0 Hz, J₂=2.5 Hz), 4.74 (d, 2H, J=3.5 Hz), 3.87 (s, 3H), 1.83 (br t, 1H, J=3.5 Hz), 1.47 (s, 6H).

### Example 13

### Synthesis of (XV)

To the vigorously stirred solution of 7-(2-methoxy-9,9-dimethyl)-fluorenyl carbinol (compound (XIV)) (4.5 g, 17.7 mmol) in Et₂O (20 ml) at 0° C. was added drop-wise concentrated HCl (9 ml, ˜5 eq.). The mixture was allowed to warm slowly to ambient and stirred for 3 hr. Phases were separated, the aqueous layer was washed again with ether and the combined organic extracts were washed with brine and evaporated under reduced pressure. The crude semi-solid chloride (4.1 g, ˜15 mmol, 85%) was dissolved in DMF (40 mL) and solid NaCN (1.5 g, 30 mmol, ˜2 eq.) was added in 3-4 portions. The mixture was stirred for 4 hr at ambient with TLC (Hexane/EtOAc 4:1) monitoring. When the reaction was complete, the reaction mixture was diluted with water and extracted with ether (3×50 mL). The combined organic extracts were—in turn—washed with water, dried over MgSO₄ and evaporated. The crystalline residue was triturated with MTBE, affording the desired pure nitrile as a white powder. The resulting compound (XV) had the following ¹H-NMR (CDCl₃) spectrum:

¹H-NMR (CDCl₃) δ (ppm)=7.62 (d, 1H, J=8.0 Hz), 7.61 (d, 1H, J=8.0 Hz), 7.35 (d, 1H, J=1.0 Hz), 7.24 (dd, 1H, J₁=8.0 Hz, J₂=1.0 Hz), 6.97 (d, 1H, J=2.5 Hz)

### Example 14

### Synthesis of (XVI)

A 25% NaOMe solution in MeOH (2.4 eq., 2.46 mmol, 0.6 ml) was added very slowly (1%/min) to the stirred cold (−25° C.) solution of 7-(2-methoxy-9,9-dimethyl)fluorenyl-acetonitrile (compound (XV)) (270 mg, 1.03 mmol) and iodine (1.2 eq., 1.23 mmol, 320 mg) in dry THF (20 ml). Only two isomers of the desired stilbene derivative could be detected on TLC (Hexane/EtOAc 4:1, R_{f}=0.55 and 0.50) at the end of reaction. The kinetic ratio between two isomers is ˜1:3 (HPLC peak area ratio, not calibrated), but the equilibrium ratio of ˜3:1 is quickly achieved (˜within 30 min for HPLC samples, dissolved in ACN) when the aliquot solution is left exposed to light.

The reaction mixture was quenched with cold water and extracted with dichloromethane (3×30 mL). The combined organic extracts were washed with aq. dilute Na₂S₂O₃, brine, dried over MgSO₄ and evaporated. The yield of the crude product, which was found pure by HPLC and NMR, was 82% (220 mg). For analytical purposes the separation of isomers was performed by trituration with Et₂O followed by filtration of the crystalline first isomer (less polar on TLC), while the more soluble semi-solid second isomer was isolated by chromatography of the filtrate evaporation residue (Hexane/EtOAc 10:1). All operations (fraction concentration on rotavapor, NMR and HPLC samples preparation etc.) were performed in the dark in order to avoid formation of the equilibrium mixture. The resulting compound (XVI) had the following ¹H-NMR (CDCl₃) spectrum:

First isomer: ¹H-NMR (CDCl₃) δ (ppm)=7.91 (d, 2H, J=2.0 Hz), 7.83 (dd, 2H, J₁=8.0 Hz, J₂=2.0 Hz), 7.75 (d, 2H, J=8.0 Hz), 7.69 (d, 2H, J=8.5 Hz), 7.00 (d, 2H, J=2.0 Hz), 6.93 (dd, 2H, J₁=8.5 Hz, J₂=2.0 Hz), 3.91 (s, 6H), 1.54 (s, 12H); ¹³C-NMR (CDCl₃) δ (ppm)=161.5, 157.3, 154.6, 143.6, 131.4, 130.6, 129.0, 124.9, 123.7, 122.6, 120.3, 118.3, 113.9, 109.2, 56.3, 47.9, 27.7

Second isomer: ¹H-NMR (CDCl₃) δ (ppm)=7.59 (d, 2H, J=8.5 Hz), 7.52 (d, 2H, J=8.0 Hz), 7.34 (dd, 2H, J₁=8.0 Hz, J₂=2.0 Hz), 7.33 (d, 2H, J=2.0 Hz), 6.91 (d, 2H, J=2.5 Hz), 6.89 (dd, 2H, J₁=8.5 Hz, J₂=2.5 Hz), 3.87 (s, 6H), 1.31 (s, 12H); ¹³C-NMR (CDCl₃) δ (ppm)=161.5, 157.1, 154.6, 131.1, 129.6, 129.2, 128.0, 124.3, 122.4, 120.3, 118.6, 113.9, 109.2, 108.3, 56.3, 47.6, 27.6

### Example 15

### Synthesis of (XVII)

4-bromoacetonitrile (2.35 g, 12.0 mmol), 4-methoxyphenylboronic acid (2.00 g, 13.2 mmol, 1.1 eq), K2PO4 (5.08 g, 23.9 mmol, 2 eq) were heated up in DMF (33 cc) to 95° C., under Ar atmosphere stream. After 20 minutes, during which water residues were removed, Pd(PPh₃)₄ ( 0.27 g, 2% eq) was added and reaction mixture was stirred for 3.5 hrs. Cool to room temperature and filter. To filtrate add chloroform (100 cc) wash with water (5×100 cc), dry (MgSO₄), filter and evaporate to yield 2.88 g solid product (85% pure by HPLC). After recrystallization from hexane:chloroform:2-propanol (205:11:9 cc) 0.74 g of light yellowish bright powder was obtained, y=28% (99% pure by HPLC). The resulting compound (XVII) has the following mass spectrum:

M.S (EI+): 223 (M⁺, 100%), 208 ([M−Me]⁺, 26%), 180 (MeO—Ph—Ph—H⁺, 22%)

### Example 16

### Synthesis of (XVIII)

A partially dissolved mixture of (4′-Methoxy-biphenyl-4-yl)-acetonitrile (compound (XVII) (0.74 g, 3.3 mmol) and iodine (0.925 g, 1.1 eq) in MTBE (8.4 cc) was stirred at −20° C., under Ar atmosphere, for 3 minutes. Sodium methoxide (25% solution in methanol, 1.7 cc, 2.2 eq) was added dropwise over 30 minutes. The orange-brown reaction mixture was allowed to reach room temperature, over 30 minutes, and stirred for additional 30 minutes. Reaction mixture was filtered and the solid was washed thoroughly with MTBE. The solid was transferred to 0.1 M sodium thiosulfate solution stirred, filtered again and washed with much water. A total of 0.53 g of 2,3-Bis-(4′-methoxy-biphenyl-4-yl)-but-2-enedinitrile were obtained at ˜90% purity (main impurity is starting material), y=33%. Adequate purity may be obtained after treating with hot P.E. and hot MeOH. The resulting compound (XVIII) has the following mass spectrum:

M.S (EI+): 442 (M⁺, 100%), 223 (MeO—Ph—Ph—CH₂CN⁺, 12%).

### Example 17

### Analysis

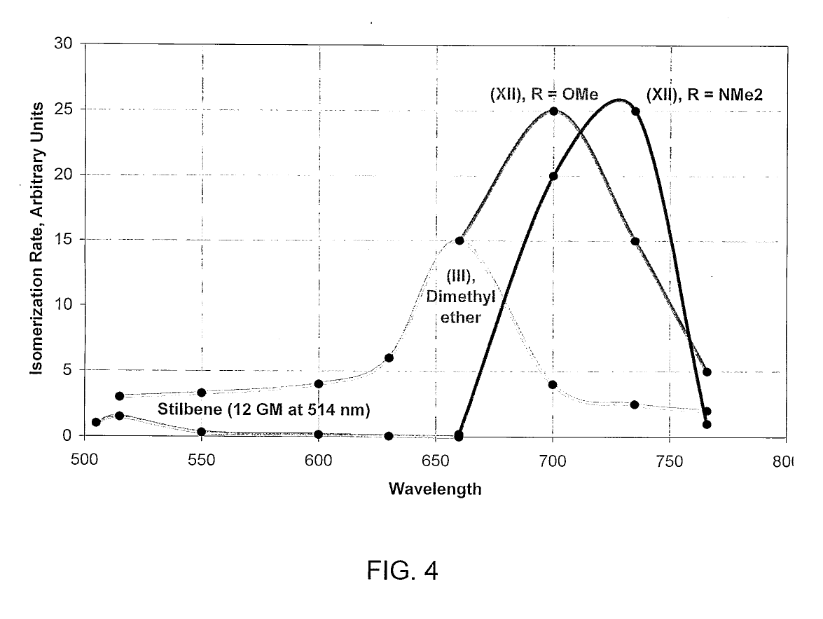

The compounds of formula XII (see Example 8), with R═OMe and NMe₂ were examined for their nonlinear photochromism. Solutions were irradiated with various wavelengths of laser light and the trans-cis isomeric ratio was followed. For comparison, identical experiments were carried out with stilbene and with the dimethyl ether of (III). The results are shown in FIG. 4. It is clear that the chromophores described here having a long conjugated system perform significantly better (a larger isomerization rate) than their counterparts with shorter conjugated bridges.

### Example 18

### Isomerization Experiment

Either four or six quartz cuvettes 10×10 mm internal dimensions, stoppered) were fixed in a line within a trough with a separation of ˜1 mm between each two cuvettes. The gaps between the cuvettes were filled with mineral oil in order to minimize internal reflections between the cuvettes when they were filled. The bank of cuvettes was placed in the path of a laser beam exiting from an OPO system, such that the beam entered the center of the first cuvette and exited from the center of the last.

The OPO laser system was based on a Nd:YAG (Continuum)-pumped OPO (Panther), that produced a train of 16 ns pulses at 10 Hz. The laser beam exiting the OPO system was filtered of stray 355 nm and 532 nm light by means of interference filters, and was collimated and shaped by use of lenses and an iris to produce a ˜2 mm diameter beam. The laser was tunable, and over its usable range the output power varied significantly. Typically 20 mW was obtained, and passage through the bank of filled cuvettes was not found to attenuate the beam significantly. Therefore, each cuvette received an identical amount of irradiation.

Solutions of the compounds to be examined were made up in amber vials in the dark at concentrations of 3 mM in an appropriate solvent. Where possible, pure trans-isomers were used, otherwise the sample with the highest trans:cis ratio was used. All subsequent handling of solutions of chromophores was carried out in the dark. To each of the cuvettes, 10 drops of a solution to be examined was added, and the volume was made up to 3 mL with ethyl acetate. The first and last cuvettes in the bank of 6 often both contained “eMMA”, and were used (a) for the normalization of different experiments, and (b) to verify that the different cuvettes all received the same amount of irradiation. Identical samples to those in the cuvettes were made up and placed alongside the cuvettes (where the beam did not pass).

The laser beam was turned on for a period of ˜16 hours, during which time the solutions in the cuvettes were irradiated and underwent 2-photon isomerization to some lesser of greater extent. At the end of the experiment the solutions in the cuvettes, and the identical samples that were placed next to them, were examined by HPLC (High-Performance Liquid Chromatography) on a C-18 reversed phase column, using an eluant gradient beginning with 50:50 MeOH:water, and reaching 100% MeOH after 5 minutes. This gradient allowed the resolution of the trans and cis isomers of the compounds. NMR spectroscopy was used to verify the attribution of peaks to isomers. In order that the chromatograms would properly reflect the ration of isomers, detection was carried out with a UV detector at a wavelength where the two isomers had the same absorption coefficient. This wavelength was found by finding the isobestic point in the absorbance spectrum of each chromophore when it was irradiated by UV light. For each chromophore this wavelength is different, and for eMMA it is 330 nm. From the results of the HPLC chromatograms, the % change in the isomeric ratio was calculated, and this value was used for the reporting of results.

In addition to experiments referenced against eMMA, experiments were performed where one of the chromophores was stilbene. This allowed some semi-quantitative analysis, since the 2-photon cross-section and quantum yield of isomerization of stilbene are well known.

Examples of experimental results include the following:

Laser wavelength = 735 nm, 16 hours irradiation

| Cuvette no. | Contents | % Change |
|---|---|---|
| 1 | eMMA | 0.5% |
| 2 | (XII); R═OMe | 3% |
| 3 | (XII); R═NMe₂ | 4.5% |
| 4 | (XII); R═CHO | 0% |

This experiment demonstrates how the extended conjugated system of (XII) tends to increase the activity of the chromophores. Where R═CHO there is an exception, because the CHO group is not a strong donor.

Laser wavelength = 700 nm, 64 hours irradiation

| Cuvette | | |
|---|---|---|
| no. | Contents | % Change |
| 1 | eMMA | 10% |
| 2 | (XII) R = OMe | 40% |
| 3 | 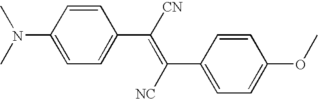 | 35% |
| 4 | (XII) R = NMe₂ | 30% |
| 5 | | |
| 6 | 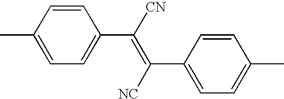 | 1% |

Again, the compounds with longer conjugated systems and stronger donors perform better. Cuvette 6 gives an example of an eMMA analog with weaker donors—as expected, it is a less active material.

Laser wavelength = 658 nm, 15 hours irradiation

| Cuvette | | % |
|---|---|---|
| no. | Contents | Change |
| 1 | eMMA | 5.0% |
| 2 | (IV) | 7.1% |
| 3 | (III) | 10.3% |
| 4 | 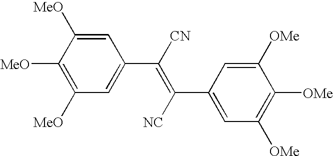 | 8.2% |
| 5 | (IV) | 10.3% |
| 6 | eMMA | 3.1% |

One may easily note that the same compound in different positions in the bank of cuvettes gives similar results. It is also clear that stronger donors (e.g. OH rather than OMe) lead to faster isomerization.

1-20. (canceled)

21. A compound of formula (1): 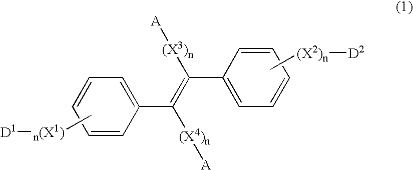
wherein
each n of (X¹)ₙ, (X²)ₙ, (X³)ₙ and (X⁴)ₙ, independently of each other, is 0, 1 or 2;
each of X¹, X², X³, and X⁴, independently of each other, is a conjugating group, each of which being selected from the group consisting of:
(i) 5- or 6-membered aromatic moieties, which may contain one, two or three heteroatoms selected from N, O and S atoms; and
(ii) C₂₋₅ alkenylenes or C₂₋₅ alkynylenes or their heteroatom chain analogs, optionally comprising N atom in the chain;
one or both of said X¹ and X² may be bound to the 3, 4 or 5 positions of the phenyl ring;
one or both of said X¹ and X² may form a fused ring with the phenyl ring at the 3-4 or 4-5 positions thereof;
D¹ and D², independently of each other, are each a donor moiety selected from the group consisting of —C₁₋₄-alkyl; —OC₁₋₄alkyl; —CH₂—O—CH₃; —SC₁₋₄-alkyl; —C₁₋₄—OH; NR′R″, wherein R′ and R″ are, independently of each other, selected from the group consisting of hydrogen, —C₁₋₄-alkyl, biphenyl, and heteroaromatic; and —CHO; and
each of —(X³)ₙ—A and —(X⁴)ₙ—A is an acceptor moiety, wherein A is selected independently from the group consisting of pyridinium, ammonium salts, —C₂₋₅-alkenyl, —C₂₋₅-alkynyl, azobenzenes, —C₁₋₄-CN, halides, —C₁₋₆—COOH or their —C₁₋₆-esters, —C₁₋₄—NO₂, —CN and —NO₂.

22. The compound according to claim 21, wherein n of (X¹)ₙ is 0 and n of (X²)ₙ is 1 or 2.

23. The compound according to claim 21, wherein n of (X¹)ₙ is 0 and n of (X²)ₙ are each 1 or 2.

24. The compound according to claim 21, wherein n of (X³)ₙ is 0 and n of (X⁴)ₙ is 1 or 2.

25. The compound according to claim 21, wherein n of (X³)ₙ and n of (X⁴)ₙ are each 1 or 2.

26. The compound according to claim 21, wherein n of (X³)ₙ and n of (X⁴)ₙ are each 0.

27. The compound according to claim 26, having the general formula (2): 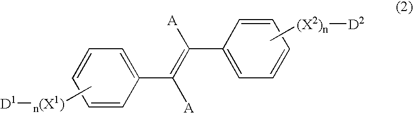
wherein one of n of (X¹)ₙ and (X²)ₙ is 0, the other n of (X²)ₙ and (X¹)ₙ is 1 or 2.

28. The compound according to claim 27, wherein each A is —CN, the n of (X²)ₙ is 1, and the n of (X¹)ₙ is 0.

29. The compound according to claim 28, wherein X² is a 5- or 6-membered aromatic moiety.

30. The compound according to claim 29, wherein said 5- or 6-membered aromatic moiety is selected from the group consisting of substituted or unsubstituted phenyl, pyridine, pyrrole, imidazole, triazole, thiophene and furan.

31. The compound according to claim 30, wherein said 6-membered aromatic moiety is phenyl.

32. The compound according to claim 28, wherein D¹ is —CH₂—O—CH₃ and D² is selected from the group consisting of —OC₁₋₄-alkyl; —NR′R″, wherein R′ and R″ are, independently of each other, selected from the group consisting of hydrogen, C₁₋₄-alkyl, biphenyl, and heteroaromatic; and —CHO.

33. The compound according to claim 32, wherein D² is —OC₁₋₄-alkyl.

34. The compound according to claim 33, wherein D² is —OCH₃.

35. The compound according to claim 32, wherein D² is —CHO.

36. The compound according to claim 32, wherein D² is —NR′R″, wherein R′ and R″ are, independently of each other, selected from the group consisting of hydrogen, C₁₋₄-alkyl, biphenyl, and heteroaromatic.

37. The compound according to claim 36, wherein R′ and R″ are the same.

38. The compound according to claim 36, wherein R′ and R″ are not the same.

39. The compound according to claim 36, wherein R′ and R″ are each C₁₋₄-alkyl.

40. The compound according to claim 36, wherein R′ and R″ are each —CH₃.

41. The compound according to claim 27, wherein each A is —CN, and the n of (X²)ₙ and the n of (X¹)ₙ are each 1.

42. The compound according to claim 41, wherein each of X¹, X² is a 5- or 6-membered aromatic moiety.

43. The compound according to claim 42, wherein said 5- or 6-membered aromatic moiety is selected from the group consisting of substituted or unsubstituted phenyl, pyridine, pyrrole, imidazole, triazole, thiophene and furan.

44. The compound according to claim 43, wherein said 6-membered aromatic moiety is phenyl.

45. The compound according to claim 42, wherein each of D¹ and D² is selected from the group consisting of —CH₂—O—CH₃; —OC₁₋₄-alkyl; —NR′R″, wherein R′ and R″ are, independently of each other, selected from the group consisting of hydrogen, C₁₋₄-alkyl, biphenyl, and heteroaromatic; and —CHO.

46. The compound according to claim 45, wherein each of D¹ and D² is —OC₁₋₄-alkyl.

47. The compound according to claim 45, wherein each of D¹ and D² is —OCH₃.

48. The compound according to claim 45, wherein each of D¹ and D² is —CH₃.

49. The compound according to claim 41, wherein each of X¹ and X² is a 5- or 6-membered aromatic moiety forming a fused ring with the phenyl ring at the 3-4 or 4-5 positions thereof.

50. The compound according to claim 49, wherein the ring system is fluorenyl.

51. The compound according to claim 27, wherein n of (X²)ₙ and n of (X¹)ₙ are each 0 and wherein one of n of (X³)ₙ and n of (X⁴)ₙ is 1 or 2.

52. The compound according to claim 51, having the general formula (3): 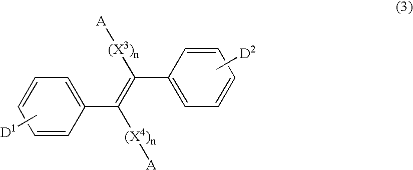
wherein one n of (X³)ₙ and (X⁴)ₙ is 1 or 2.

53. The compound according to claim 52, wherein each of D¹ and D² is selected from the group consisting of C₁₋₄-alkyl; —OC₁₋₄-alkyl; —CH₂—O—CH₃; —SC₁₋₄-alkyl; —C₁₋₄—OH; NR′R″, wherein R′ and R″ are, independently of each other, selected from the group consisting of hydrogen, C₁₋₄-alkyl, biphenyl, and heteroaromatic; and —CHO.

54. The compound according to claim 53, wherein each of D¹ and D² is a —OC₁₋₄-alkyl.

55. The compound according to claim 52, wherein
each of D¹ and D² is a —OC₁₋₄-alkyl,
A is selected from the group consisting of pyridinium, ammonium salts, —C₂₋₅-alkenyl, —C₂₋₅-alkynyl, azobenzenes, —C₁₋₄—CN, halides, —C₁₋₆—COOH or their —C₁₋₆-esters, —C₁₋₄—NO₂, —CN and —NO₂,
each of X³ and X⁴ is selected from the group consisting of:
(i) 5- or 6-membered aromatic moieties which may contain one, two or three heteroatoms selected from the group consisting of N, O and S atoms; and
(ii) —C₂₋₅ alkenylenes or —C₂₋₅ alkynylenes or their heteroatom chain analogs optionally comprising N atom in the chain; and
n=1.

56. The compound according to claim 55, wherein each of D¹ and D² is a —OC₁₋₄-alkyl, A is selected from the group consisting of —C₁₋₄—CN, C₁₋₆—COOH or their C₁₋₆-esters, C₁₋₄—NO₂, —CN and —NO₂ and each of X³ and X⁴ is a C₂₋₅ alkenylene or C₂₋₅ alkynylene.

57. The compound according to claim 56, wherein each of D¹ and D² is a —OC₁₋₄-alkyl, A is —CN and each of X³ and X⁴ is a C₂₋₅ alkenylene or C₂₋₅ alkynylene.

58. The compound according to claim 57, wherein each of D¹ and D² is —OCH₃, A is —CN and each of X³ and X⁴ is C₂-alkynylene.

59. A compound selected from the group consisting of:
2-(4′-Methoxy-biphenyl-4-yl)-3-(4-methoxymethyl-phenyl)-but-2-enedinitrile;
2-(4′-Formyl-biphenyl-4-yl)-3-(4-methoxymethyl-phenyl)-but-2-enedinitrile;
2-(4′-Dimethylamino-biphenyl-4-yl)-3-(4-methoxymethyl-phenyl)-but-2-enedinitrile;
2,3-Bis-(4′-methoxy-biphenyl-4-yl)-but-2-enedinitrile; and
2,3-Bis-(4′-methyl-biphenyl-4-yl)-but-2-enedinitrile.

60. A compound selected from the group consisting of:

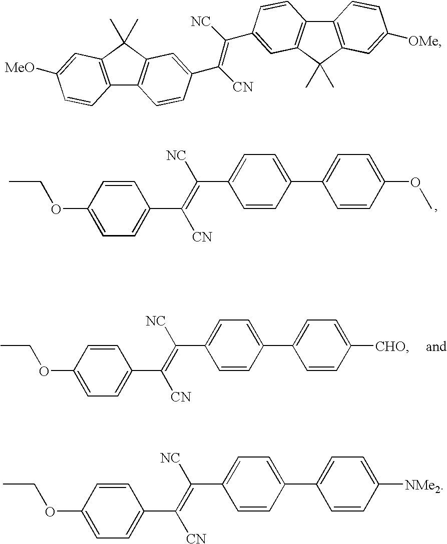

61. A compound selected from the group consisting of the compounds herein designated (VIII), (IX), (X), (XI), and (XII).

62. A process for the preparation of a compound of formulae (XI) and (XII) in accordance with the following Scheme 1: 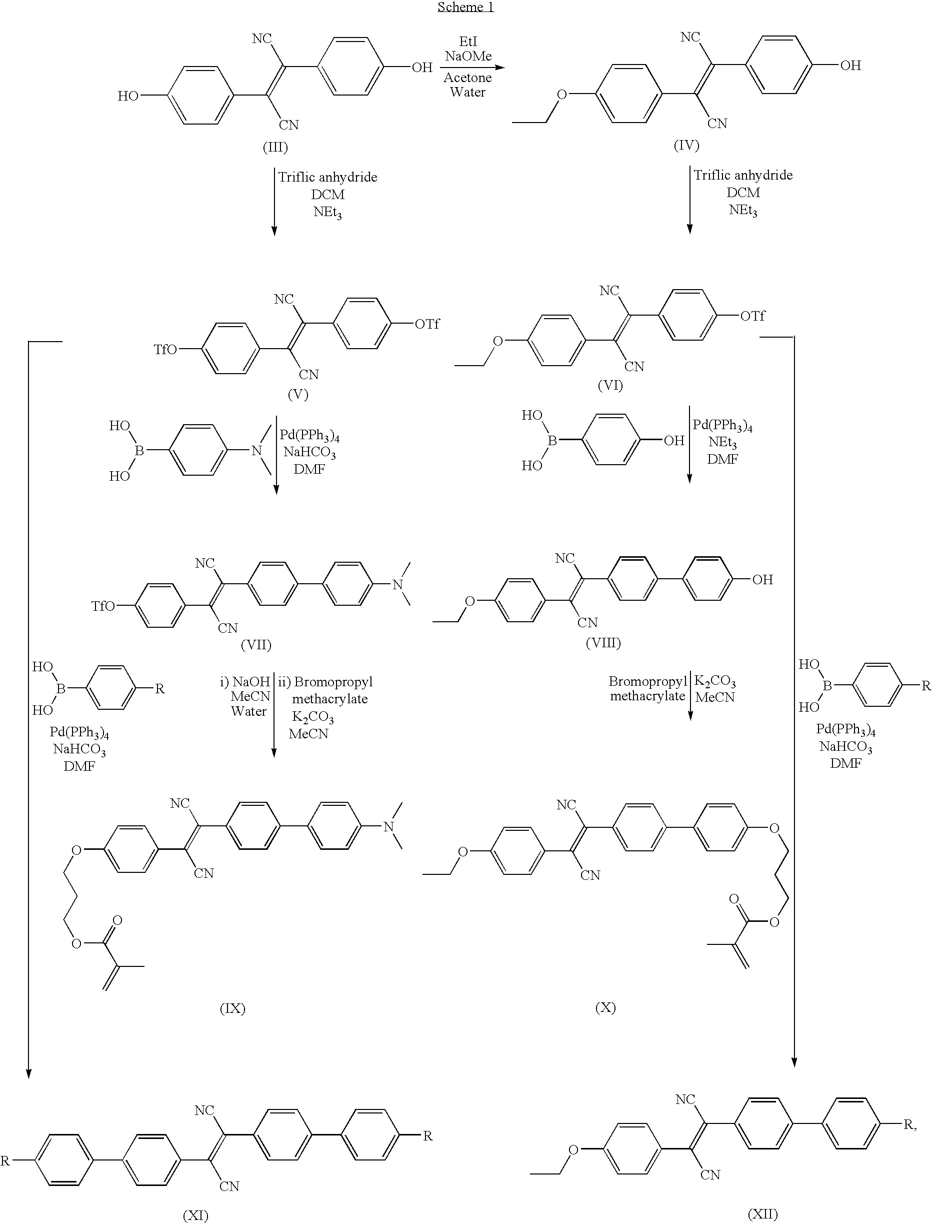
wherein each R, independently of each other, may be —OMe or —NMe₂.

63. A process for the preparation of a compound of formula (XVIII) in accordance with the following scheme:

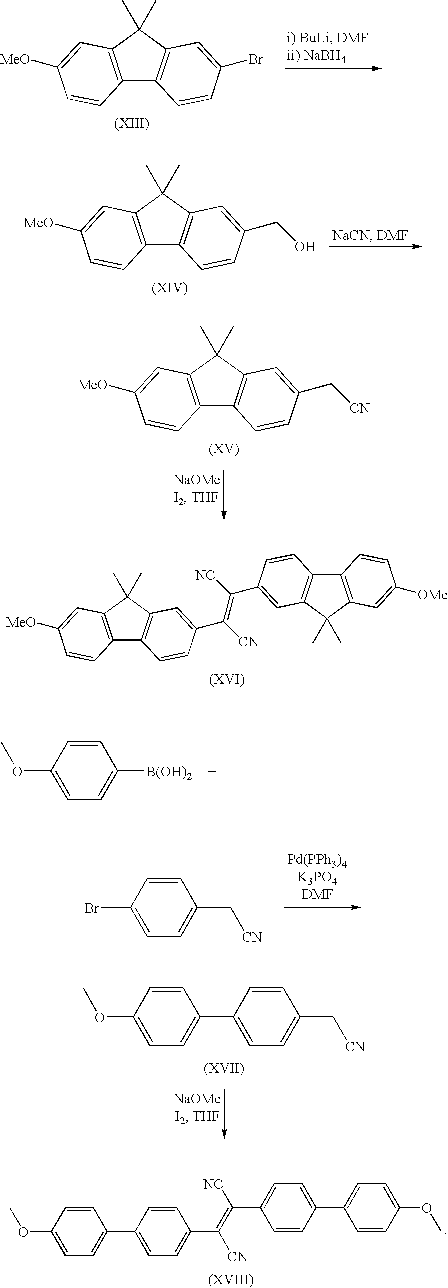

64. A process for the preparation of a compound of formula (XI) in accordance with the following scheme:

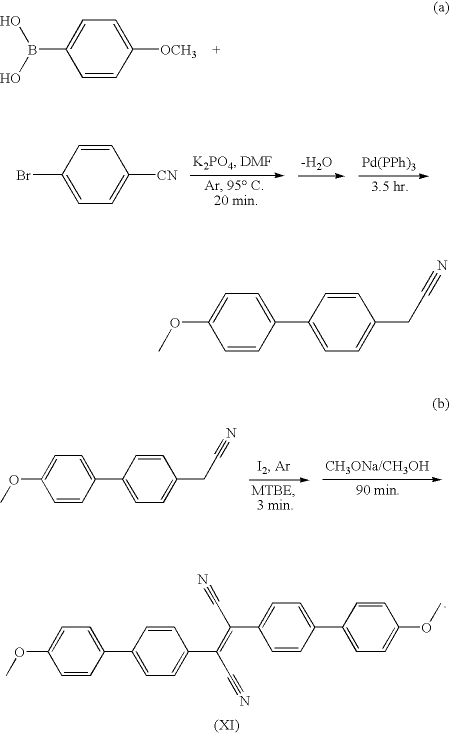

65. A compound of claim 21 for use in nonlinear media.

66. A compound of claim 21 for use as dyes, reference standards, chromophoric optical medium, optical limiters, components in microfabriation systems, nanotechnological devices, or targeted medical therapeutic applications.

67. A compound of claim 21 for use as the chromophoric optical medium of a 3-dimensional optical memory.

68. A device comprising a compound of claim 21.

69. A device according to claim 68, selected from the group consisting of chromophoric optical medium, optical limiters, components in microfabrication systems, nanotechnological devices.

70. A formulation comprising a compound of claim 21.

71. A formulation according to claim 70 being a dye, a reference standard, or a pharmaceutical composition.

72. A compound prepared according to claim 62.

73. A formulation comprising a compound according to claim 72.

74. A device comprising a compound according to claim 72.

75. A compound according to claim 21 substituted by at least one polymerizable group.

76. The compound according to claim 75, being a monomer in the preparation of polymers.

77. The compound according to claim 75, wherein said polymerizable group is selected from the group consisting of methylacrylate and methyl methacrylate.

78. A monomer being a compound according to claim 21, having a polymerizable group.

79. The compound according to claim 75 for use in the production of plastics.

80. A compound according to claim 21, being in an isomeric form selected from cis, trans, E and Z forms, wherein said isomeric form is of the central stilbene double bond.
